(19) United States
(12) Patent Application Publication
(10) Pub. No.: US 2004/0009912 A1
(43) Pub. Date: Jan. 15, 2004

(54) Method for treating schizoprhenia and related phychoes, and the use of erythropoietin or erythropoietin derivatives for treating schizophrenic disorders and related pyschoes

(76) Inventor: Hannelore Ehrenreich, Gottingen (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/363,617

(22) PCT Filed: Sep. 4, 2001
(86) PCT No.: PCT/EP01/10198

(30) Foreign Application Priority Data
Sep. 4, 2000 (DE) 100434576

(51) Int. Cl. A61K 38/22

(52) U.S. Cl. 514/12

(57) ABSTRACT

Method for treatment and/or prophlaxis of schizophrenia and related psychoses of a mammal, erythropoietin being administered to the mammal.

The present invention relates to a method for treating schizophrenia and related psychoses, subsumed in the following under “schizophrenia”, and also a means for treating schizophrenia in the sense of the above definition using erythropoietin.

The aetiology and pathogenesis of schizophrenia in the sense of the above definition are to date unknown. In fact there is agreement about the important role of genetic influences, however a series of presumably relevant co-factors is known, for example neurotrauma, drug consumption etc. which appear to have an influence on the outbreak of the disease. In particular the molecular and cellular mechanisms which play a role pathogenetically in this disease are also unknown. No really good animal models for schizophrenia have therefore been produced to date. The available animal models cover merely partial aspects of the disease.

It is therefore the object of the present invention to make available a method for treating schizophrenia and related psychoses and also a means for treating schizophrenia and related psychoses.

This object is achieved by the method according to patent claim 1 and also by use according to patent claim 8 or 17. Advantageous developments of the method according to the invention and uses according to the invention are presented in the respective dependent claims.

There is thereby understood by schizophrenia actual schizophrenia and related psychoses. By treatment there should be considered not only the treatment in the case of symptoms which have already occurred but also the prophylactic use in the case of people who are particularly at risk, for example in the case of people with a high hereditary factor, with neurotrauma, psychotrauma and the like.

The proposed medicinal neuroprotection in schizophrenia is thereby a generally completely new therapeutic and prophylactic starting point. This takes into account the observation confirmed again and again by epidemiologists and clinicians that, already in the course of the first episode of the disease, a dramatic impairment of cognitive/mental function happens which is at least partially irreversible and levels off generally in the further episodes rather on a constant or a less progressive level. Here now the consistent use of a neuroprotective therapy approach intervenes also as “add-on therapy” in conjunction with a symptom-arresting neuroleptic, particularly in the first episode of the psychosis. A preventive use of erythropoietin in the people particularly at risk is also proposed according to this invention.

Erythropoietin, also described in brief as “EPO”, is a glycoprotein occurring naturally in the body with a molecular weight of 34,000 dalton (W. Jelkmann, “Erythropoietin: Structure, Control of Production, and Function”, Physiological Reviews, 1992, Volume 72, pages 449 to 489). It is an essential growth factor for the production of erythrocytes and was isolated for the first time already in 1977.

Erythropoietin has been in frequent clinincal use for many years in nephrodialysis in the case of patients who have renal anaemia, for obtaining fairly large quantities of autologous blood before planned operations and it also hit the press headlines as a sports drug.

Erythropoietin thereby proved to be exceptionally well tolerated. In particular, the frequently therapeutically desired stimulation of the haematopoiesis with polyglobulin and also an arterial hypertonia which is rarely to be seen should be mentioned as a relevant side-effect. Both effects are to be expected mainly after chronic erythropoietin administration. These are remedied when required relatively simply by medicinal treatment or blood-letting. Intolerance reactions or anaphylactic reactions in the case of erythropoietin constitute rarities.

As a fairly large protein with a molecular weight of approximately 34,000 dalton, erythropoietin is not able as is known generally to surmount the blood-brain barrier. A directly intracerebroventricular administration of erythropoietin, i.e. direct infusion of erythropoietin into the brain tissue is ruled out however in human beings usually, because of risks which are involved in the installation and the maintenance of a temporary ventricular drainage, such as infections or haemorrhages.

An advantageous starting point for the method according to the invention and for the use of erythropoietin according to the invention is that, in the case of schizophrenia, a disorder of the blood-brain barrier generally occurs in the acute stage of the psychosis, which permits a speedy crossing of the erythropoietin circulating in the blood into the brain.

The present invention now proceeds advantageously here in that it has been detected that, in the case of an acute phase of a psychosis, the blood-brain barrier is permeable for erythropoietin. Hence a systemic peripheral administration, for example parenterally as well as vascularly, intranasally, per inhalation, in particularly intravenously, subcutaneously and/or intramuscularly (for example for depot type ), of erythropoietin is nevertheless successful.

In the case of a chronic treatment or long-term therapy, a slow effect of the administration of erythropoietin takes place as long as, with persistently high blood levels (by administration of up to 200,000 IU per week) of erythropoietin, a crossing of erythropoietin via the blood-brain barrier into the brain takes place nevertheless despite the intact blood-brain barrier.

Due to this surprising crossing of erythropoietin into the brain in the case of the acute phase of a psychosis or because of very high blood levels of erythropoietin during chronic treatment, erythropoietin can be used in order to influence all three mechanisms potentially involved in the pathogenesis of the schizophrenia, said mechanisms resulting in a neuronal dysfunction: these are apoptosis, the metabolic disorder of the nerve cells and also the synaptic junctions/axon sprouting.

It can be established in summary that medicinal neuroprotection in the case of schizophrenia is generally a completely new therapeutic and prophylactic approach.

A few examples of the methods according to the invention and the results thereof are shown in the following.

There are shown:

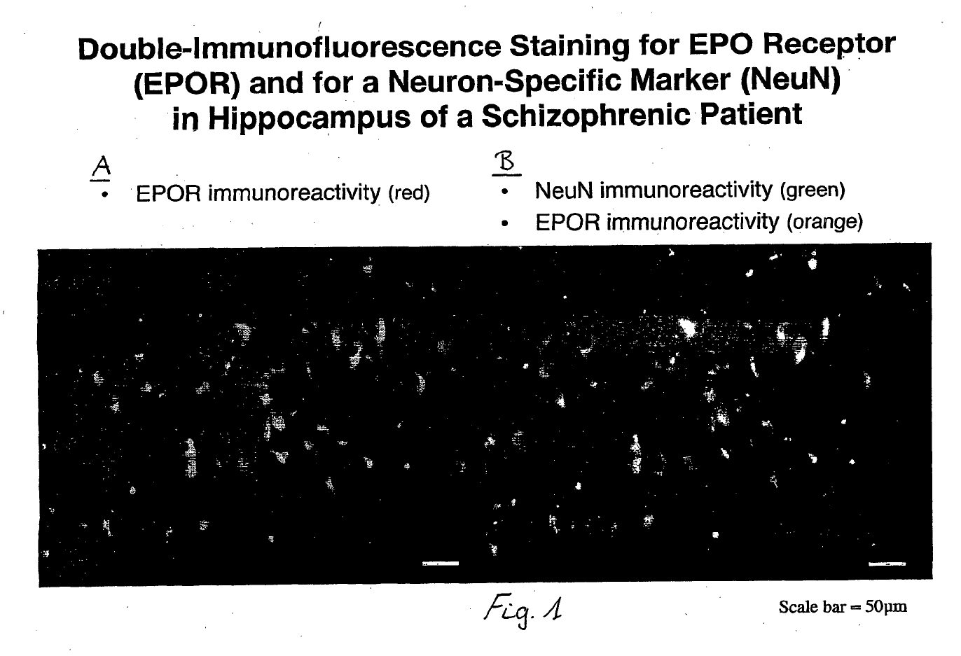

FIG. 1A and FIG. 1B immunohistochemical examinations of sections of the brain of schizophrenics.

In a test series on three schizophrenics in the subacute illness stage (first or second manifestation) and on two healthy patients, there was administered intravenously on one occasion to the individual patients respectively 40,000 i.U indium-111-erythropoietin with in total 120-185 MBq. Subsequently, single photon emission computer tomographies (SPECT-pictures) were taken, the pictures being produced 4.18 to 21 or 42 to 45 hours after administration of the radioactively marked erythropoietin.

The subsequent Table 1 shows the quotients “average impulse content of the brain/average impulse content of the bone marrow of the skull cap”.

| | 4 h | 18-21 h | 42-45 h |
|---|---|---|---|
| Experimentee 1 | 0.69 | 0.49 | 0.46 |
| Experimentee 2 | 0.61 | 0.47 | 0.43 |
| Patient 1 | 0.75 | 0.59 | 0.58 |
| Patient 2 | 0.80 | 0.56 | 0.50 |
| Patient 3 | 0.76 | 0.64 | 0.49 |

In the three patients 1 to 3 with schizophrenia, a clear intracerebral accumulation of the radioactively marked erythropoietin is shown at all three points in time, this accumulation being globally higher in the patients 1 to 3 than in the healthy experimentees 1 and 2. This multi-accumulation of the radioactive erythropoietin can be seen immediately from the quotients shown in Table 1.

It has been shown for the first time hence that erythropoietin surmounts the blood-brain barrier more strongly in the case of schizophrenics in the (sub)acute phase of the psychosis than in the case of healthy people. Furthermore, it has been established for the first time that erythropoietin is able to surmount this even in the case of healthy people with an intact blood-brain barrier with a correspondingly high dosage. Hence a chronic treatment of schizophrenics, even beyond the acute illness phase, i.e. even in the case of a blood-brain barrier which is intact again, can effect sufficiently high intracerebral levels as neuroprotective add-on therapy.

Erythropoietin is thereby able to influence all three mechanisms potentially involved in the pathogenesis of the schizophrenia, said mechanisms resulting in a neuronal dysfunction;

a) apoptosis;

b) metabolic disorder of the nerve cells;

c) synaptic junctions/axon sprouting.

The precondition for the neuroprotective effect of erythropoietin in the brain of schizophrenics, which is sought after here, is its bonding to specific erythropoietin receptors on nerve cells. These were detected for the first time in the immunohistochemical studies carried out for the present invention.

FIG. 1 shows a histological section from the hippocampus of a schizophrenic (post mortem brain) in which, by means of immunohistochemistry, erythropoietin receptors (EPOR: red colouration, left image, FIG. 1A) and by means of a double fluorescence method, the localisation of the same could be detected on nerve cells (EPOR: orange colouration, NeuN=nerve cell marker; green colouration, right image, FIG. 1B). It can be detected immediately that the green fluorescent nerve cells are also marked with red fluorescent EPOR antibodies so that an orange colouration is produced in FIG. 1B. It has been established for the first time with this study that nerve cells in the brain of schizophrenics have immunoreactivity for erythropoietin receptors.

For FIGS. 1A and 1B, the sections were deparaffinated in Hemo-DE (Fischer Scientific, Schwerte, Germany), three washing steps were implemented for 5 minutes, they were rehydrated in a decreasing alcohol sequence, washed with distilled water, boiled in citrate buffer, washed in tris buffered common salt solution (TBS), incubated with 10% blocking serum in 0.05% Tween-20/TBS at room temperature and subsequently incubated with a polyclonal hare-anti-human EPOR antibody (1:200, C-20, Santa Cruz Biotechnology, Heidelberg, Germany) in 2% goat serum/PBS at 4° C. overnight. After washing in 0.05% Tween-20/TBS, the sections were incubated with Texas Red-marked goat-anti-hare antibodies (1:100, Vector Laboratories Inc., Burlingame, Calif., U.S.A.) in a humidity chamber (30 min). After washing with 0.05% Tween-20/TBS, the sections were incubated with a monoclonal mouse-antineuronal core (NeuN) antibody (1:500, Chemcon Int. Inc., Temecula, Calif., U.S.A.) in 2% horse serum/PBS at +4° C. (24 h), washed in 0.05% Tween-20/TBS and incubated in a humidity chamber with horse-anti-mouse antibodies marked with fluoresceine (FITC) (1:100, vector) for 30 min. The sections were then washed in 0.05% Tween-20/TBS and TBS and finally embedded in Vectashield (vector) fluorescence medium.

1. Method for treatment and/or prophylaxis of schizophrenia and related psychoses of a mammal, characterised in that erythropoietin is administered to the mammal.

2. Method according to the preceding claim, characterised in that the administration is effected parenterally/systemically.

3. Method according to the preceding claim, characterised in that the administration is effected vascularly, intranasally and/or per inhalation.

4. Method according to one of the two preceding claims, characterised in that the administration is effected intravenously, subcutaneously and/or intramuscularly.

5. Method according to one of the preceding claims, characterised in that erythropoietin is administered to a human being.

6. Method according to one of the preceding claims, characterised in that erythropoietin is administered. in the acute phase of a schizophrenia or related psychosis.

7. Method according to one of the preceding claims, characterised in that erythropoietin is administered in a high dose over a fairly long period of time.

8. Use of erythropoietin for producing a drug for treatment and/or prophylaxis of schizophrenia and related pyschoses.

9. Use according to the preceding claim for producing a drug to be administered parenterally/ systemically.

10. Use according to one of the two preceding claims for producing a drug to be administered peripherally.

11. Use according to one of the three preceding claims for producing a drug to be administered vascularly.

12. Use according to the preceding claim for producing a drug to be administered intravenously.

13. Use according to one of the claims 8 to 12, in a dose of 5,000 IU to 200,000 IU per administration and/or per day and/or per week.

14. Use according to the preceding claim, in a dose of 35,000 IU per administration and/or day.

15. Use according to one of the claims 8 to 14, characterised in that a native or recombinant human or animal erythropoietin or a derivative thereof is used as erythropoietin.

16. Use according to one of the claims 8 to 15, characterised in that the mammal is a human being.

17. Use of erythropoietin as means for treatment and/or prophylaxis of schizophrenia and related psychoses of a mammal.

18. Use according to the preceding claim as means to be administered parenterally/ systemically.

19. Use according to the preceding claim as means to be administered vascularly.

20. Use according to the preceding claim as means to be administered intravenously.

21. Use according to one of the claims 17 to 20 for treating schizophrenia and related psychoses.

22. Use. according to one of the claims 17 to 21, in a dose of 5,000 IU to 2000,000 IE per administration and/or per day and/or per week.

23. Use according to one of the claims 17 to 22, in a dose of 35,000 IU per administration and/or day.

24. Use according to one of the claims 17 to 23, characterised in that a native or recombinant human or animal erythropoietin or a derivative thereof is used as erythropoietin.

25. Use according to one of the claims 17 to 24, characterised in that the mammal is a human being.
